(19) United States
(12) Patent Application Publication
(10) Pub. No.: US 2025/0049872 A1
(43) Pub. Date: Feb. 13, 2025

(54) hypoallergenic milk with immune enhancing function and preparation method thereof

(71) Applicant: BEIDAHUANG WANDA MOUNTAINS DAIRY CO.,LTD, Harbin (CN)

(72) Inventors: Yang ZHENG, Harbin (CN); Chao LIANG, Harbin (CN); Jie XIA, Harbin (CN); Zhandong WANG, Harbin (CN); Qingyun WANG, Harbin (CN)

(21) Appl. No.: 18/564,997

(22) PCT Filed: Jun. 26, 2023
(86) PCT No.: PCT/CN2023/102281

§ 371 Date: Nov. 28, 2023

(30) Foreign Application Priority Data
Oct. 18, 2022 (CN) 202211276970.5

(51) Int. Cl. A61K 36/185; A23C 9/12; A23C 9/13; A23C 9/133; A61K 31/375; A61K 31/716; A61K 38/39; A61K 38/47; A61P 37/08

(52) U.S. Cl. CPC A61K 36/185; A23C 9/1206; A23C 9/1307; A23C 9/1322; A23C 9/133; A61K 31/375; A61K 31/716; A61K 38/39; A61K 38/47; A61P 37/08; C12Y 302/01108

(57) ABSTRACT

The present disclosure relates to a hypoallergenic milk with immune enhancing function and a preparation method thereof. To solve the problem of milk allergy and lactose intolerance populations being unable to intake milk nutrition through traditional dairy products, the present disclosure provides a hypoallergenic milk with immune enhancing function, including raw milk, collagen peptide, and yeast β-glucan, acerola cherry powder, maltitol, lactase, stabilizer, essence and water; and the raw milk is A2β-casein organic raw milk. The hypoallergenic milk provided by the present disclosure reduces allergenic substances and risk in terms of formula composition and production process, especially suitable for lactose intolerance and A1β-casein allergy populations. The present disclosure further adopts a triple immune science formula, retaining the active ingredients and high-quality nutrients in milk. Long term consumption can effectively improve human immunity, and is suitable for people who need to supplement milk nutrition and enhance immunity.

### CROSS-REFERENCE

This application is a continuation of International Application No. PCT/CN2023/102281 with a filling date of Jun. 26, 2023, designating the United States, now pending, and further claims to the benefit of priority from Chinese Application No. 202211276970.5 with a filing date of Oct. 18, 2022. The content of the aforementioned applications, including any intervening amendments thereto, are incorporated herein by reference.

### TECHNICAL FIELD

The present disclosure relates to the technical field of modified milk processing, in particular to a hypoallergenic milk with immune enhancing function and preparation method thereof.

### BACKGROUND

With the continuous improvement of the living standards of the Chinese people, the requirements for beverages are also increasing, and the nutritional value of food is more and more important. Dairy products are a natural food with complete nutritional components, appropriate proportions, easy digestion and absorption, and high nutritional value. Milk is a very important food in dietary structure of people, and has significant effects in preventing various chronic diseases and promoting the growth of various human functions. Considering that the average intake of dairy products by urban and rural residents in China is insufficient, thus, the “Dietary Guidelines for Chinese Residents” strongly advocates drinking milk to improve the nutritional and health status of Chinese residents. The “Chinese Balanced Diet Pagoda” recommends that each person should consume 300 grams of milk and dairy products per day.

Milk allergy is an abnormal immune reaction caused by ingestion of milk or food containing dairy products. It causes a wide range of adverse reactions in children under 3 years of age, and its incidence rate is as high as 7.5%. In recent years, with the continuous increase in the consumption of milk and dairy products among the population, the milk allergy rate has also gradually increased, and milk allergy has become a food safety issue that cannot be ignored. However, there is currently no comprehensive and effective cure for milk allergic diseases at home and abroad. The only effective way to prevent milk allergies is to avoid consuming foods containing milk and dairy products.

In addition, milk contains a special sugar called lactose, which has various physiological functions such as promoting intelligence, promoting intestinal health, and promoting mineral absorption. However, some people lack lactase in their bodies, which makes it difficult to digest and decompose the lactose in milk, leading to digestive discomfort such as abdominal distension and diarrhea. This is known as lactose intolerance. Therefore, this group of people cannot consume milk and dairy products, causing the loss of opportunities to intake milk nutrients.

Therefore, there is an urgent demand for a type of hypoallergenic milk on the market to meet the needs of milk allergy and lactose intolerant populations for milk nutrition intake.

### SUMMARY

To solve the problem of milk allergy and lactose intolerant populations being unable to intake milk nutrition through traditional dairy products, the present disclosure provides a hypoallergenic milk with immune enhancing function and a preparation method thereof.

The technical solution of the present disclosure are shown as below:

A hypoallergenic milk with immune enhancing function, including every 1000 kg of hypoallergenic milk contains 809-908 kg of raw milk, 24.7-42.2 kg of collagen peptide, 0.5-1.0 kg of yeast β-glucan, 5.7-29.5 kg of acerola cherry powder, 5-11 kg of maltitol, 0.1-1.3 kg of lactase, 1-4 kg of stabilizer, 0-2 kg of food essence, and 55-100 kg of water, wherein the raw milk is A2β-casein organic raw milk.

Further, each 250 mL of the hypoallergenic milk contains not more than 1.25 g of lactose, not less than 12.6 g of protein, not less than 96 mg of vitamin C, and not less than 87.5 mg of β-glucan.

Further, a protein content of the A2β-casein organic raw milk is not be less than 3.12 g/100 g, and a β-casein content of the A2β-casein organic raw milk is not less than 0.9 g/100 g.

Further, the collagen peptide is sourced from bovine bone with a protein content not less than 90 g/100 g.

Further, a vitamin C content of the acerola cherry powder is not less than 16 g/100 g.

Further, the lactase is sterile with an enzyme activity not less than 7500 U/g.

Further, the stabilizer is composed of microcrystalline cellulose, gellan gum, carrageenan, and sodium carboxymethyl cellulose, and a mass ratio of the microcrystalline cellulose, the gellan gum, the carrageenan, and the sodium carboxymethyl cellulose is 0.3-1.7:0.4-0.9:0.2-0.8:0.1-0.6.

Further, a preparation method of hypoallergenic milk with immune enhancing function, including the following steps:
step 1, conducting degassing, centrifugal clarification, homogenization, and pasteurization to the raw milk in sequence to obtain pasteurized milk, then refrigerating and storing at a temperature of 2-6° C.;
step 2, taking some of the pasteurized milk obtained from step 1, rising the temperature to 55-70° C., adding the collagen peptide, the yeast β-glucan, the maltitol, and the stabilizer in a stirred state, and mixing and dissolving for 10-15 minutes;
step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 5-10 minutes to mix and dissolve all raw and auxiliary materials evenly;
step 4, filtering and cooling the material solution obtained from step 3 below 20° C., mixing the cooled material solution with a remaining pasteurized milk for 15-20 minutes, diluting to volume and then stirring for 15-20 minutes, and then conducting homogenization and sterilization, wherein a pressure of the homogenization is 20±5 MPa, a temperature of the homogenization is 65±5° C.; a temperature of ultra-high temperature sterilization is 135-150° C., and time of the ultra-high temperature sterilization is 2-15 seconds;
step 5, lowering the temperature of the material solution obtained in step 4 to below 25° C., then adding sterile lactase, and then conducting aseptic filling.

Further, 9. the temperature of the pasteurization in step 1 is 75-85° C. and the time is 10-15 seconds.

Further, adding the food essence before diluting to volume in step 4.

The advantageous effects of the present disclosure:

The hypoallergenic milk provided by the present disclosure reduces allergenic substances in terms of formula composition and production process, and further adopts a triple immune science formula, retaining the active ingredients and high-quality nutrients in the milk, which can improve the absorption rate of the body, and long-term use can effectively improve human immunity. The hypoallergenic milk provided by the present disclosure is rich in nutrients with a pleasant essence, and has the characteristics of reducing allergy risk and improving body immunity. The hypoallergenic milk provided by the present disclosure is suitable for people who needs supplement milk nutrition to drink, especially for lactose intolerance and A1β-casein allergy populations, providing a wider range of dairy product choices for susceptible population, which has important practical significance and social value.

### DETAILED DESCRIPTION OF THE EMBODIMENTS

The following is a further explanation of the technical solution of the present disclosure in conjunction with the embodiments, but not limited to this. Any modification or equivalent substitution of the technical solution of the present disclosure without departing from the spirit and scope of the technical solution should be included in the scope of the present disclosure. The process equipment or devices not specifically specified in the following embodiments are all conventional equipment or devices in the field. If not specifically specified, the raw materials used in the embodiments of the present disclosure can be commercially available; If not specifically specified, the technical means used in the embodiments of the present disclosure are all conventional means well-known to those skilled in the art.

### Embodiment 1

In the present embodiment, a hypoallergenic milk with immune enhancing function is provided, and every 1000 kg of the hypoallergenic milk contains 885 kg of raw milk, 36 kg of collagen peptide, 1 kg of yeast β-glucan, 6.4 kg of acerola cherry powder, 7 kg of maltitol, 0.3 kg of lactase, 2.3 kg of stabilizer, 0.5 kg of milk essence, 1.5 kg of Hami melon essence and 60 kg of water. Wherein the stabilizer is composed of 1 kg of microcrystalline cellulose, 0.5 kg of gellan gum, 0.5 kg of carrageenan, and 0.3 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

Research has found that the A2β-casein in the milk produces β-casomorphin-7 during the digestion process in the body, β-casomorphin-7 will interact with various systems in the body, resulting in a series of negative effects during digestion. And A2β-casein milk does not produce β-casomorphin-7 during the digestion process, so that it can alleviate gastrointestinal discomfort caused by milk protein intolerance in some populations. The milk source of A2β-casein is very rare, with only 30% of milk cows being purebred A2 type cows, and all the milk they produce is A2β-casein.

This embodiment selects healthy milk cows, and collects their hair, tissue, or body fluids to extract their DNA. After strict genetic testing technology screening, the milk cows which produces all the β-casein is A2β-casein are purebred A2 type cows. The purebred A2 type cows are selected from the other cows to a separated area for individual feeding and activities to obtain A2β-casein organic raw cow milk.

In the present embodiment, the collagen peptide is sourced from bovine bone with a protein content not less than 90 g/100 g. The manufacturer is Hubei Nutratide Biotech Co., Ltd.

Vitamin C is an essential ingredient in maintaining human life, enhancing the ability to resist stress and immunity to the external environment. High concentrations of vitamin C help reduce cystine in food proteins to L-cysteine, which contributes to synthesize antibodies and enhances the body immunity. In addition, vitamin C also has detoxification, antioxidant and other effects. Acerola cherry is a natural source of vitamin C, and the vitamin C content of the acerola cherry powder used in the hypoallergenic milk with immune enhancing function is up to 16%. Due to the acidity of vitamin C, when the acidity is below 4.6, milk protein undergoes a denaturation reaction. In this embodiment, acerola cherry powder is pre-dissolved in water to reduce its acidity, and then mixed with other materials to avoid protein denaturation and ensure product quality. The vitamin C content of the acerola cherry powder used in the present embodiment is not less than 16 g/100 g.

Yeast β-glucan was officially approved by the Health Department on 20 May 2010 as a new food ingredient, which is produced from brewer's yeast through the steps of extraction, acid and alkali treatment, spray drying and so on. Yeast β-glucan is a polysaccharide with β-1,3-D-glucan as the main chain and β-1,6-D-glucan as the branched chain. By specifically binding to the macrophage surface receptor, β-glucan activates the macrophage to secretes IL, α-IFN, and TNF to regulate the immunity.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 75° C. for 15 seconds, then refrigerating and storing at a temperature of 4° C.;
Step 2, taking some of the pasteurized milk obtained from step 1, rising the temperature to 65° C., adding the collagen peptide, the yeast β-glucan, the maltitol, and the stabilizer in a stirred state, and mixing and dissolving for 15 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 10 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 15 minutes,
accurately weighing the milk essence and the Hami melon essence to add into the medium storage tank, then using the pasteurized milk diluting to volume and then stirring for 15 minutes, and then conducting homogenization and sterilization, wherein the pressure of the homogenization is 20 MPa, the temperature of the homogenization is 65° C.; the temperature of ultra-high temperature sterilization is 139° C., and the time of the ultra-high temperature sterilization is 4 seconds;
Step 5, lowering the temperature of the material solution obtained in step 4 to below 25° C., then adding sterile lactase, and then conducting aseptic filling.

This embodiment reduces the risk of allergens in terms of raw material selection and process treatment, and achieves the effect of improving the body immunity in terms of ingredient ratio and formula synergy. The product is rich in nutrients and has a pleasant essence, suitable for consumption by susceptible populations and those who need to enhance their immunity.

### Embodiment 2

In the present embodiment, a hypoallergenic milk with immune enhancing function is provided, and every 1000 kg of the hypoallergenic milk contains 908 kg of raw milk, 24.7 kg of collagen peptide, 0.5 kg of yeast β-glucan, 5.7 kg of acerola cherry powder, 5 kg of maltitol, 0.1 kg of lactase, 1 kg of stabilizer, and 55 kg of water. Wherein the stabilizer is composed of 0.3 kg of microcrystalline cellulose, 0.4 kg of gellan gum, 0.2 kg of carrageenan, and 0.1 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 80° C. for 15 seconds, then refrigerating and storing at a temperature of 3° C.;
Step 2, taking some of the pasteurized milk obtained from step 1, rising the temperature to 55° C., adding the collagen peptide, the yeast β-glucan, the maltitol, and the stabilizer in a stirred state, and mixing and dissolving for 10 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 5 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 20 minutes, using the pasteurized milk diluting to volume and then stirring for 15 minutes, and then conducting homogenization and sterilization, wherein the pressure of the homogenization is 25 MPa, the temperature of the homogenization is 60° C.; the temperature of ultra-high temperature sterilization is 135° C., and the time of the ultra-high temperature sterilization is 15 seconds;
Step 5, lowering the temperature of the material solution obtained in step 4 to below 25° C., then adding sterile lactase, and then conducting aseptic filling.

### Embodiment 3

In the present embodiment, a hypoallergenic milk with immune enhancing function is provided, and every 1000 kg of the hypoallergenic milk contains 880 kg of raw milk, 42.2 kg of collagen peptide, 0.6 kg of yeast β-glucan, 6 kg of acerola cherry powder, 6 kg of maltitol, 0.6 kg of lactase, 3 kg of stabilizer, 1 kg of milk essence, 0.6 kg of strawberry essence and 60 kg of water. Wherein the stabilizer is composed of 1.7 kg of microcrystalline cellulose, 0.9 kg of gellan gum, 0.2 kg of carrageenan, and 0.2 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 80° C. for 10 seconds, then refrigerating and storing at a temperature of 2° C.;
Step 2, taking some of the pasteurized milk obtained from step 1, rising the temperature to 70° C., adding the collagen peptide, the yeast β-glucan, the maltitol, and the stabilizer in a stirred state, and mixing and dissolving for 12 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 7 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 17 minutes,
accurately weighing the milk essence and the strawberry essence to add into the medium storage tank, then using the pasteurized milk diluting to volume and then stirring for 20 minutes, and then conducting homogenization and sterilization, wherein the pressure of the homogenization is 15 MPa, the temperature of the homogenization is 70° C.; the temperature of ultra-high temperature sterilization is 150° C., and the time of the ultra-high temperature sterilization is 2 seconds;
Step 5, lowering the temperature of the material solution obtained in step 4 to below 25° C., then adding sterile lactase, and then conducting aseptic filling.

### Embodiment 4

In the present embodiment, a hypoallergenic milk with immune enhancing function is provided, and every 1000 kg of the hypoallergenic milk contains 885 of raw milk, 37.5 kg of collagen peptide, 0.5 kg of yeast β-glucan, 5.7 kg of acerola cherry powder, 11 kg of maltitol, 0.7 kg of lactase, 2.6 kg of stabilizer, 0.5 kg of milk essence, 0.5 kg of banana essence and 55 kg of water. Wherein the stabilizer is composed of 0.3 kg of microcrystalline cellulose, 0.9 kg of gellan gum, 0.8 kg of carrageenan, and 0.6 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 75° C. for 10 seconds, then refrigerating and storing at a temperature of 6° C.;
Step 2, taking some of the pasteurized milk obtained from step 1, rising the temperature to 60° C., adding the collagen peptide, the yeast β-glucan, the maltitol, and the stabilizer in a stirred state, and mixing and dissolving for 14 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 8 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 18 minutes,
accurately weighing the milk essence and the banana essence to add into the medium storage tank, then using the pasteurized milk diluting to volume and then stirring for 19 minutes, and then conducting homogenization and sterilization, wherein the pressure of the homogenization is 22 MPa, the temperature of the homogenization is 62° C.; the temperature of ultra-high temperature sterilization is 137° C., and the time of the ultra-high temperature sterilization is 4 seconds;
Step 5, lowering the temperature of the material solution obtained in step 4 to below 25° C., then adding sterile lactase, and then conducting aseptic filling.

### Embodiment 5

In the present embodiment, a hypoallergenic milk with immune enhancing function is provided, and every 1000 kg of the hypoallergenic milk contains 828 kg of raw milk, 35 kg of collagen peptide, 0.8 kg of yeast β-glucan, 25 kg of acerola cherry powder, 7.5 kg of maltitol, 0.7 kg of lactase, 1.5 kg of stabilizer, 1 kg of milk essence, 0.5 kg of cream essence and 100 kg of water. Wherein the stabilizer is composed of 0.4 kg of microcrystalline cellulose, 0.6 kg of gellan gum, 0.2 kg of carrageenan, and 0.3 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 85° C. for 10 seconds, then refrigerating and storing at a temperature of 5° C.;
Step 2, taking some of the pasteurized milk obtained from step 1, rising the temperature to 65° C., adding the collagen peptide, the yeast β-glucan, the maltitol, and the stabilizer in a stirred state, and mixing and dissolving for 12 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 5 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 19 minutes,
accurately weighing the milk essence and the cream essence to add into the medium storage tank, then using the pasteurized milk diluting to volume and then stirring for 18 minutes, and then conducting homogenization and sterilization, wherein the pressure of the homogenization is 24 MPa, the temperature of the homogenization is 65° C.; the temperature of ultra-high temperature sterilization is 135° C., and the time of the ultra-high temperature sterilization is 10 seconds;
Step 5, lowering the temperature of the material solution obtained in step 4 to below 25° C., then adding sterile lactase, and then conducting aseptic filling at 20° C. after stirring evenly.

### Embodiment 6

In the present embodiment, a hypoallergenic milk with immune enhancing function is provided, and every 1000 kg of the hypoallergenic milk contains 841 kg of raw milk, 31.3 kg of collagen peptide, 0.6 kg of yeast β-glucan, 18 kg of acerola cherry powder, 6.5 kg of maltitol, 1.1 kg of lactase, 1 kg of stabilizer, 0.5 kg of Hami melon essence and 100 kg of water. Wherein the stabilizer is composed of 0.3 kg of microcrystalline cellulose, 0.4 kg of gellan gum, 0.2 kg of carrageenan, and 0.1 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 75° C. for 15 seconds, then refrigerating and storing at a temperature of 3° C.;
Step 2, taking some of the pasteurized milk obtained from step 1, rising the temperature to 57° C., adding the collagen peptide, the yeast β-glucan, the maltitol, and the stabilizer in a stirred state, and mixing and dissolving for 13 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 7 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 20 minutes,
accurately weighing the Hami melon essence to add into the medium storage tank, then using the pasteurized milk diluting to volume and then stirring for 19 minutes, and then conducting homogenization and sterilization, wherein the pressure of the homogenization is 25 MPa, the temperature of the homogenization is 68° C.; the temperature of ultra-high temperature sterilization is 140° C., and the time of the ultra-high temperature sterilization is 8 seconds;
Step 5, lowering the temperature of the material solution obtained in step 4 to below 25° C., then adding sterile lactase, and then conducting aseptic filling.

### Embodiment 7

In the present embodiment, a hypoallergenic milk with immune enhancing function is provided, and every 1000 kg of the hypoallergenic milk contains 809 kg of raw milk, 42.2 kg of collagen peptide, 1 kg of yeast β-glucan, 29.5 kg of acerola cherry powder, 11 kg of maltitol, 1.3 kg of lactase, 4 kg of stabilizer, 0.8 kg of concentrated milk essence, 1.2 kg of vanilla essence and 100 kg of water. Wherein the stabilizer is composed of 1.7 kg of microcrystalline cellulose, 0.9 kg of gellan gum, 0.8 kg of carrageenan, and 0.6 kg of sodium carboxymethyl cellulose.

In the present embodiment, each 250 mL of the hypoallergenic milk contains not more than 1.25 g of lactose, not less than 12.6 g of protein, not less than 96 mg of vitamin C, and not less than 87.5 mg of β-glucan.

The raw milk used in the present embodiment is A2β-casein organic raw milk, the protein content of the A2β-casein organic raw milk is not be less than 3.12 g/100 g, and the β-casein content of the A2β-casein organic raw milk is not less than 0.9 g/100 g.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

In the present embodiment, the lactase is sterile, and the enzyme activity ≥7500 U/g.

The present embodiment provides a preparation method of hypoallergenic milk with immune enhancing function, including the following steps:
Step 1, conducting degassing, centrifugal clarification, homogenization, and pasteurization to the raw milk in sequence to obtain pasteurized milk, wherein the pasteurization at a temperature of 75° C. for 15 seconds, then refrigerating and storing at a temperature of 4° C.;
Step 2, taking some of the pasteurized milk obtained from step 1, rising the temperature to 63° C., adding the collagen peptide, the yeast β-glucan, the maltitol, and the stabilizer in a stirred state, and mixing and dissolving for 15 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 10 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 15 minutes,
accurately weighing the concentrated milk essence and the vanilla essence to add into the medium storage tank, then using the pasteurized milk diluting to volume and then stirring for 20 minutes, and then conducting homogenization and sterilization, wherein the pressure of the homogenization is 20 MPa, the temperature of the homogenization is 70° C.; the temperature of ultra-high temperature sterilization is 142° C., and the time of the ultra-high temperature sterilization is 10 seconds;
Step 5, lowering the temperature of the material solution obtained in step 4 to below 25° C., then adding sterile lactase, and then conducting aseptic filling.

### Comparative Group 1

This comparative group provides a type of modified milk, using ordinary raw milk as the raw material. The specific formula and preparation method are as follows:

In the comparative group, a hypoallergenic milk with immune enhancing function is provided, and every 1000 kg of the hypoallergenic milk contains 885 kg of raw milk, 36 kg of collagen peptide, 1 kg of yeast β-glucan, 6.4 kg of acerola cherry powder, 7 kg of maltitol, 2.3 kg of stabilizer, 0.5 kg of milk essence, 1.5 kg of Hami melon essence and 60.3 kg of water. Wherein the stabilizer is composed of 1 kg of microcrystalline cellulose, 0.5 kg of gellan gum, 0.5 kg of carrageenan, and 0.3 kg of sodium carboxymethyl cellulose.

In the present embodiment, the collagen peptide is sourced from bovine bone with a protein content not less than 90 g/100 g. The manufacturer is Hubei Nutratide Biotech Co., Ltd.

The vitamin C content of the acerola cherry powder used in the present embodiment is not less than 16 g/100 g.

The modified milk provided by the comparative group includes the following steps:
Step 1, conducting degassing, centrifugal clarification, homogenization, and pasteurization to the raw milk in sequence to obtain pasteurized milk, wherein the pasteurization at a temperature of 75° C. for 15 seconds, then refrigerating and storing at a temperature of 4° C.;
Step 2, taking some of the pasteurized milk obtained from step 1, rising the temperature to 65° C., adding the collagen peptide, the yeast β-glucan, the maltitol, and the stabilizer in a stirred state, and mixing and dissolving for 20 minutes;
Step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 10 minutes to mix and dissolve all raw and auxiliary materials evenly;
Step 4, filtering the material solution obtained from step 3 through 80 mesh filter cloth and cooling below 20° C. through cold exhaust, poring the cooled material solution into a medium storage tank to mix with the remaining pasteurized milk for 15 minutes,
accurately weighing the milk essence and the Hami melon essence to add into the medium storage tank, then using the pasteurized milk diluting to volume and then stirring for 20 minutes, and then conducting homogenization and sterilization, wherein the pressure of the homogenization is 25 MPa, the temperature of the homogenization is 65° C.; the temperature of ultra-high temperature sterilization is 142° C., and the time of the ultra-high temperature sterilization is 10 seconds, to guarantee the temperature not be lower than 134° C., and conducting aseptic filling.

### Effect Validation Test 1:

Functional validation was conducted using the hypoallergenic milk for enhancing immunity prepared in Embodiment 1. It was recommended to drink 100 g of the hypoallergenic milk for enhancing immunity prepared in Embodiment 1 once a day. \textcircled{1} Freeze dry the hypoallergenic milk for enhancing immunity prepared in Embodiment 1, with a powder yield of 19.5%. \textcircled{2} SPF grade ICR mice were selected for the experiment, and 160 healthy female mice weighing 18-22 g were randomly divided into 4 immune groups with 40 mice in each group. \textcircled{3} If the adult weight was calculated as 60 kg, the sample dosage was equivalent to 0.325 g/kg bw, and the high, medium, and low dose groups were set to 1.63 g/kg bw, 3.25 g/kg bw, and 9.75 g/kg bw, respectively. At the same time, a negative control group was set up. Animals were gavaged at 2% of their body weight, and the experimental group solvent was distilled water. \textcircled{4} After continuous gavage for 30 days, various immune indicators were measured. The first immunized group was conducted carbon clearance test and animal organ/body ratio test. The second immunized group was conducted half hemolytic value (HC₅₀) test and plaque-forming cell assay (Jerne modified slide method) to measure various immune indicators. The third immunized group was conducted delayed type hypersensitivity (DTH). The fourth immunized group was conducted mice peritoneal macrophage phagocytosis test for chicken red blood cells. And the fifth immunized group was subjected to ConA induced mice spleen lymphocyte transformation experiment and NK cell activity assay.

### 1. The Effect on Body Weight of Mice

The effect of 30 days of administration of hypoallergenic milk for enhancing immunity prepared in Embodiment 1 on the body weight of mice is shown in the table below.

TABLE 1
Effect of hypoallergenic milk for enhancing immunity prepared in Embodiment
1 on body weight of mice (the first immunized group) (X̅ ± SD)

| | Dosage | Number of | Initial body | F | | Final body | F | |
|---|---|---|---|---|---|---|---|---|
| Group | (g/kg · bw) | animals | weight (g) | value | P | weight (g) | value | P |
| Control | — | 10 | 19.43 ± 0.83 | | | 32.73 ± 1.12 | | |
| group | | | | | | | | |
| Low dose | 1.63 | 10 | 19.77 ± 1.16 | 1.306 | >0.05 | 33.28 ± 1.07 | 0.812 | >0.05 |
| group | | | | | | | | |
| Medium | 3.25 | 10 | 19.17 ± 0.88 | | | 33.06 ± 1.37 | | |
| dose group | | | | | | | | |
| High dose | 9.75 | 10 | 19.37 ± 0.89 | | | 32.57 ± 1.02 | | |
| group | | | | | | | | |

TABLE 2
Effect of hypoallergenic milk for enhancing immunity prepared in Embodiment
1 on body weight of mice (the second immunized group) (X̅ ± SD)

| | Dosage | Number of | Initial body | F | | Final body | F | |
|---|---|---|---|---|---|---|---|---|
| Group | (g/kg · bw) | animals | weight (g) | value | P | weight (g) | value | P |
| Control | — | 10 | 19.24 ± 0.82 | | | 33.42 ± 0.86 | | |
| group | | | | | | | | |
| Low dose | 1.63 | 10 | 19.15 ± 0.54 | | | 33.78 ± 1.08 | | |
| group | | | | | | | | |
| Medium | 3.25 | 10 | 19.61 ± 0.98 | 1.156 | >0.05 | 33.17 ± 1.06 | 0.521 | >0.05 |
| dose | | | | | | | | |
| group | | | | | | | | |
| High dose | 9.75 | 10 | 19.43 ± 1.15 | | | 33.78 ± 1.20 | | |
| group | | | | | | | | |

TABLE 3
Effect of hypoallergenic milk for enhancing immunity prepared in Embodiment
1 on body weight of mice (the third immunized group) (X̅ ± SD)

| | Dosage | Number of | Initial body | F | | Final body | F | |
|---|---|---|---|---|---|---|---|---|
| Group | (g/kg · bw) | animals | weight (g) | value | P | weight (g) | value | P |
| Control group | — | 10 | 19.26 ± 0.86 | | | 33.27 ± 0.93 | | |
| Low dose | 1.63 | 10 | 19.18 ± 0.97 | | | 32.91 ± 1.11 | | |
| group | | | | | | | | |
| Medium dose | 3.25 | 10 | 19.34 ± 0.66 | 0.407 | >0.05 | 33.00 ± 1.14 | 0.338 | >0.05 |
| group | | | | | | | | |
| High dose | 9.75 | 10 | 19.55 ± 0.92 | | | 32.86 ± 0.62 | | |
| group | | | | | | | | |

TABLE 4
Effect of hypoallergenic milk for enhancing immunity prepared in Embodiment
1 on body weight of mice (the fourth immunized group) (X̅ ± SD)

| | Dosage | Number of | Initial body | F | | Final body | F | |
|---|---|---|---|---|---|---|---|---|
| Group | (g/kg · bw) | animals | weight (g) | value | P | weight (g) | value | P |
| Control | — | 10 | 19.43 ± 1.08 | | | 33.47 ± 1.09 | | |
| group | | | | | | | | |
| Low dose | 1.63 | 10 | 19.08 ± 0.76 | 0.879 | >0.05 | 32.65 ± 1.11 | 1.856 | >0.05 |
| group | | | | | | | | |
| Medium | 3.25 | 10 | 19.60 ± 0.96 | | | 34.06 ± 0.96 | | |
| dose group | | | | | | | | |
| High dose | 9.75 | 10 | 19.34 ± 0.78 | | | 33.20 ± 1.26 | | |
| group | | | | | | | | |

TABLE 5
Effect of hypoallergenic milk for enhancing immunity prepared in Embodiment
1 on body weight of mice (the fifth immunized group) (X̅ ± SD)

| | Dosage | Number of | Initial body | F | | Final body | F | |
|---|---|---|---|---|---|---|---|---|
| Group | (g/kg · bw) | animals | weight (g) | value | P | weight (g) | value | P |
| Control | — | 10 | 19.17 ± 0.63 | | | 33.48 ± 1.42 | | |
| group | | | | | | | | |
| Low dose | 1.63 | 10 | 19.56 ± 1.06 | 2.226 | >0.05 | 33.13 ± 1.39 | 1.766 | >0.05 |
| group | | | | | | | | |
| Medium dose | 3.25 | 10 | 19.39 ± 0.67 | | | 32.34 ± 0.80 | | |
| group | | | | | | | | |
| High dose | 9.75 | 10 | 19.23 ± 0.96 | | | 32.79 ± 1.03 | | |
| group | | | | | | | | |

From the comparison of data in Table 1 to Table 5, it can be seen that there were no significant differences in the initial and final body weights between the low, medium, and high dose groups of animals and the control group (P>0.05), indicating that the hypoallergenic milk prepared in Embodiment 1 under the experimental conditions had no effect on the weight growth of mice.

### 2. The Effect on Organ to Body Weight Ratio in Mice

The effect of 30 days of administration of hypoallergenic milk for enhancing immunity prepared in Embodiment 1 on organ to body weight ratio in mice is shown in Table 6.

TABLE 6
Effect of hypoallergenic milk for enhancing immunity prepared in Embodiment
1 on organ to body weight ratio in mice (X̅ ± SD)

| | Dosage | Number of | Spleen/body | F | | Thymus/body | F | |
|---|---|---|---|---|---|---|---|---|
| Group | (g/kg·· bw) | animals | weight ratio | value | P | weight ratio | value | P |
| Control group | — | 10 | 0.46 ± 0.02 | | | 0.33 ± 0.02 | | |
| Low dose | 1.63 | 10 | 0.42 ± 0.07 | 1.166 | >0.05 | 0.32 ± 0.02 | 2.919 | >0.05 |
| group | | | | | | | | |
| Medium dose | 3.25 | 10 | 0.43 ± 0.05 | | | 0.32 ± 0.07 | | |
| group | | | | | | | | |
| High dose | 9.75 | 10 | 0.43 ± 0.06 | | | 0.30 ± 0.03 | | |
| group | | | | | | | | |

From Table 6, it can be seen that the spleen to body weight ratio and thymus to body weight ratio of the hypoallergenic milk for enhancing immunity prepared in Embodiment 1, were orally administered to mice at different doses for 30 days. There were no significant differences between the low, medium, and high dose groups and the control group (P>0.05), indicating that the hypoallergenic milk prepared in Embodiment 1 has no effect on the organ to body weight ratio of mice.

### 3. The Effect on Delayed Type Hypersensitivity (DTH) in Mice

The effect of 30 days of administration of the hypoallergenic milk for enhancing immunity prepared in Embodiment 1 on delayed type hypersensitivity (DTH) in mice is shown in Table 7.

TABLE 7
Effect of the hypoallergenic milk for enhancing immunity prepared in Embodiment
1 on delayed type hypersensitivity (DTH) in mice (X̅ ± SD)

| | | | | | Footpad | | | |
|---|---|---|---|---|---|---|---|---|
| | | | Footpad | | thickness | Difference in | | |
| | | | thickness | | after 24 | footpad | | |
| | | | before | | hours from | thickness | | |
| | | | SRBC | | SRBC | before and | | |
| | Dosage | Number of | injection | | injection | after injection | F | |
| Group | (g/kg · bw) | animals | (mm) | P | (mm) | (mm) | value | P |
| Control group | — | 10 | 2.12 ± 0.08 | | 2.60 ± 0.13 | 0.48 ± 0.08 | | |
| Low dose | 1.63 | 10 | 2.11 ± 0.08 | >0.05 | 2.70 ± 0.09 | 0.59 ± 0.06 | 6.854 | <0.01 |
| group | | | | | | | | |
| Medium dose | 3.25 | 10 | 2.13 ± 0.05 | | 2.76 ± 0.05 | 0.63 ± 0.05** | | |
| group | | | | | | | | |
| High dose | 9.75 | 10 | 2.13 ± 0.11 | | 2.79 ± 0.11 | 0.66 ± 0.10** | | |
| group | | | | | | | | |

Note: **There is a highly significant difference (P<0.01) compared to the control group.

From Table 7, it can be seen that the hypoallergenic milk for enhancing immunity prepared in Embodiment 1 was orally administered to mice at different doses for 30 days. After statistical analysis, the difference in footpad swelling (footpad thickening) before and after injection of SRBC was found, and there was a highly significant difference (P<0.01) between the medium and high dose groups and the control group. This indicates that the hypoallergenic milk prepared in Embodiment 1 can enhance the delayed type hypersensitivity of mice.

### 4. The Effect on Splenic Lymphocyte Transformation in Mice

The effect of 30 days of administration of the hypoallergenic milk for enhancing immunity prepared in Embodiment 1 on the transformation of mouse spleen lymphocytes is shown in Table 8.

TABLE 8
Effect of the hypoallergenic milk for enhancing
immunity prepared in Embodiment 1 on splenic
lymphocyte transformation in mice (X̅ ± SD)

| | Dosage | Number of | | F | |
|---|---|---|---|---|---|
| Group | (g/kg · bw) | animals | OD difference | value | P |
| Control group | — | 10 | 0.295 ± 0.042 | 0.428 | >0.05 |
| Low dose group | 1.63 | 10 | 0.297 ± 0.027 | | |
| Medium dose | 3.25 | 10 | 0.301 ± 0.012 | | |
| High dose | 9.75 | 10 | 0.304 ± 0.015 | | |

group

From Table 8, it can be seen that the hypoallergenic milk for enhancing immunity prepared in Embodiment 1 was orally administered to mice at different doses for 30 days. After statistical analysis, there was no significant difference in the splenic lymphocyte transformation function of mice in each dose group compared to the control group (P>0.05), indicating that the hypoallergenic milk for enhancing immunity prepared in Embodiment 1 did not enhance the splenic lymphocyte transformation function of mice.

### 5. The Effect on the Half Hemolytic Value (HC₅₀) in Mice

The effect of 30 days of administration of the hypoallergenic milk for enhancing immunity prepared in Embodiment 1 on the half hemolytic value (HC₅₀) of mice is shown in Table 9.

TABLE 9
Effect of the hypoallergenic milk for enhancing immunity
prepared in Embodiment 1 on the
half hemolytic value (HC₅₀) of mice (X̅ ± SD)

| | Dosage | Number of | | F | |
|---|---|---|---|---|---|
| Group | (g/kg · bw) | animals | Sample HC₅₀ | value | P |
| Control group | — | 10 | 156.8 ± 13.25 | 8.171 | <0.01 |
| Low dose | 1.63 | 10 | 173.2 ± 10.24* | | |
| group | | | | | |
| Medium dose | 3.25 | 10 | 184.8 ± 20.18** | | |
| group | | | | | |
| High dose | 9.75 | 10 | 189.3 ± 17.54** | | |
| group | | | | | |

Note:
*There is a significant difference of P < 0.05 compared to the control group,
**a highly significant difference of P < 0.01 compared to the control group.

From Table 9, it can be seen that the half hemolytic value (HC₅₀) of the medium and high dose groups of the hypoallergenic milk for enhancing immunity prepared in Embodiment 1 was highly significantly different from the control group after oral administration of different doses to mice for 30 days (P<0.01). The half hemolytic value (HC₅₀) of the low dose group was significantly different from the control group (P<0.05), indicating that the hypoallergenic milk prepared in Embodiment 1 can increase the half hemolytic value (HC₅₀) of mice.

### 6. The Effect on Antibody Producing Cells in Mice

The effect of 30 days of administration of the hypoallergenic milk for enhancing immunity prepared in Embodiment 1 on antibody producing cells in mice is shown in Table 10.

TABLE 10
Effect of the hypoallergenic milk for enhancing
immunity prepared in Embodiment 1 on
antibody producing cells in mice (X̅ ± SD)

| | | | Number of | | |
|---|---|---|---|---|---|
| | Dosage | Number of | plaques | F | |
| Group | (g/kg · bw) | animals | (/10⁶ splenic cells) | value | P |
| Control | — | 10 | 19.28 ± 1.64 | 1.178 | >0.05 |
| group | | | | | |
| Low dose | 1.63 | 10 | 20.33 ± 1.47 | | |
| group | 3.25 | 10 | 19.03 ± 2.25 | | |
| Medium | | | | | |
| dose group | | | | | |
| High dose | 9.75 | 10 | 19.38 ± 1.34 | | |
| group | | | | | |

From Table 10, it can be seen that the hypoallergenic milk for enhancing immunity prepared in Embodiment 1 was orally administered to mice for 30 days, and there was no significant difference in the number of plaques between each dose group and the control group (P>0.05), indicating that the hypoallergenic milk for enhancing immunity prepared in Embodiment 1 did not enhance the function of antibody producing cells in mice.

### 7. The Effect on Carbon Clearance Function in Mice

The effect of 30 days of administration of the hypoallergenic milk for enhancing immunity prepared in Embodiment 1 on the carbon clearance ability of mice is shown in Table 11.

TABLE 11
Effect of the hypoallergenic milk for enhancing immunity
prepared in Embodiment 1 on carbon
clearance function in mice (X̅ ± SD)

| | Dosage | Number of | Phagocytic | | |
|---|---|---|---|---|---|
| Group | (g/kg · bw) | animals | index (a) | F value | P |
| Control group | — | 10 | 4.95 ± 0.85 | 6.537 | <0.01 |
| Low dose group | 1.63 | 10 | 5.34 ± 0.77 | | |
| Medium dose | 3.25 | 10 | 5.78 ± 0.28** | | |
| group | | | | | |
| High dose group | 9.75 | 10 | 6.02 ± 0.31** | | |

Note:
**There is a highly significant difference (P < 0.01) compared to the control group.

From Table 11, it can be seen that the hypoallergenic milk for enhancing immunity prepared in Embodiment 1 was orally administered to mice at different doses for 30 days. After statistical analysis, there was a significant difference in phagocytic index between the medium and high dose groups and the control group (P<0.01), indicating that the hypoallergenic milk for enhancing immunity prepared in Embodiment 1 can increase the carbon clearance function of mice.

### 8. The Effect of Phagocytosis of Chicken Red Blood Cells by Mouse Peritoneal Macrophagess

The effect of 30 days of administration of the hypoallergenic milk for enhancing immunity prepared in Embodiment 1 to mice on the phagocytosis of chicken red blood cells by mouse peritoneal macrophages is shown in Table 12.

TABLE 12
Effect of the hypoallergenic milk for enhancing immunity prepared in Embodiment 1 on the phagocytosis
of chicken red blood cells by mouse peritoneal macrophages (X̅ ± SD)

| | Dosage | Number of | Phagocytosis | Transformed | Phagocytic | F | |
|---|---|---|---|---|---|---|---|
| Group | (g/kg · bw) | animals | rate (%) | variables | index | value | P |
| Control group | — | 10 | 22.5 ± 2.99 | 28.3 ± 2.05 | 0.390 ± 0.049 | | |
| Low dose | 1.63 | 10 | 24.6 ± 3.60 | 29.7 ± 2.43 | 0.428 ± 0.047 | | |
| Medium dose | 3.25 | 10 | 25.1 ± 2.13 | 30.0 ± 1.39 | 0.431 ± 0.048 | 5.206 | <0.01 |
| group | | | | | | | |
| High dose | 9.75 | 10 | 27.0 ± 3.23** | 31.3 ± 2.16** | 0.471 ± 0.039** | | |

Note:
**There is a highly significant difference (P < 0.01) compared to the control group.

From Table 12, it can be seen that thehypoallergenic milk for enhancing immunity prepared in Embodiment 1 was orally administered to mice at different doses. After 30 days of statistical processing, the four groups of phagocytosis rates were tested for homogeneity of variance, and the variance was not uniform. The variable transformation X=Sin−1\sqrt{p} was performed, wherein P is the phagocytosis rate, expressed as a decimal. The original data of the four groups were conducted variable transformation and the variance was uniform, then comparing the results of the four groups. The phagocytic index of the high dose group showed a highly significant difference compared to the control group (P<0.01). This indicates that the hypoallergenic milk prepared in Embodiment 1 can enhance the phagocytic function of mouse peritoneal macrophages.

### 9. The Effect on NK Cell Activity in Mice

TABLE 13
Effect of the hypoallergenic milk for enhancing
immunity prepared in Embodiment
1 on NK cell activity in mice (X̅ ± SD)

| | | Number | NK cell | | | |
|---|---|---|---|---|---|---|
| | Dosage | of | activity | Transformed | F | |
| Group | (g/kg · bw) | animals | (%) | variables | value | P |
| Control | — | 10 | 25.0 ± 2.78 | 30.0 ± 1.84 | 1.180 | >0.05 |
| group | | | | | | |
| Low dose | 1.63 | 10 | 25.4 ± 1.83 | 30.2 ± 1.45 | | |
| group | | | | | | |
| Medium | 3.25 | 10 | 23.7 ± 0.75 | 29.3 ± 0.71 | | |
| dose group | | | | | | |
| High dose | 9.75 | 10 | 24.6 ± 1.94 | 29.5 ± 1.63 | | |
| group | | | | | | |

From Table 13, it can be seen that the hypoallergenic milk for enhancing immunity prepared in Embodiment 1 was orally administered to mice for 30 days. The variable transformation X=Sin−1\sqrt{p} of NK cell activity was conducted, wherein P is NK cell activity, expressed as a decimal. After statistical analysis, there was no significant difference in NK cell activity between each dose group and the control group (P>0.05). This indicates that the hypoallergenic milk prepared in Embodiment 1 for enhancing immunity has no effect on the NK cell activity in mice.

### Experimental Conclusion

The hypoallergenic milk for enhancing immunity prepared in Embodiment 1 was orally administered to mice at different doses for 30 days, and four types of tests were conducted, totaling 7 tests, including cellular immune function (ConA induced mouse spleen lymphocyte transformation test (MTT method) and delayed type hypersensitivity test (footpad thickening method), humoral immune function (antibody producing cell test (modified slide method) and test of half hemolytic value (HC₅₀)), test of monocyte macrophage phagocytic function (mice carbon clearance test and test of mice peritoneal macrophage phagocytizing chicken red blood cell) and NK cell activity assay. The results showed that the hypoallergenic milk prepared in Embodiment 1 of the medium and high dose groups could significantly enhance the delayed type hypersensitivity and carbon clearance phagocytic index of mice. The hypoallergenic milk prepared in Embodiment 1 of the low, medium, and high dose groups could enhance the half hemolytic value (HC₅₀) of mice, while the high dose group could enhance the phagocytic function of peritoneal macrophages. According to the standard of criterion in the “Functional Evaluation Methods of Health Food”, it is believed that the hypoallergenic milk prepared in Embodiment 1 has the function of enhancing immunity.

The results indicate that A2β-casein functional milk of Embodiment 1 can significantly enhance delayed type hypersensitivity and carbon clearance phagocytic index in mice, increase the half hemolytic value (HC₅₀) of mice, and enhance the phagocytic function of peritoneal macrophages. Therefore, A2β-casein functional milk of Embodiment 1 has the function of enhancing immunity.

### Effect Validation Test 2:

Tests were conducted on Embodiment 1 and the comparative group 1.

Through clinical symptoms of lactose intolerance and urine galactose test, 60 subjects with lactose intolerance between the ages of 18-55 years were selected, regardless of gender. Randomly divided into three groups: experimental group, control group 1, and control group 2. The experimental group was divided into four stages: washout period, the first intervention period, washout period, and the second intervention period. The control group consisted of two stages: washout period and intervention period. The experimental period is 30 days, with a washout period of 10 days and an intervention period of 5 days. The experimental group consumed 100 mL of the sample in Embodiment 1 daily in the first intervention period, 100 mL of the sample in comparative group 1 daily in the second intervention period; the control group 1 consumed 100 mL of the sample in Embodiment 1 daily in the intervention period; and the control group 2 consumed 100 mL of the sample in comparative group 1 daily in the intervention period. The subjects were observed daily for any abnormal symptoms such as diarrhea, abdominal distension, nausea, vomiting, etc. During the experiment, visual analogue scale (VAS), average defecation frequency and consistency, serum inflammation, and immune biomarkers were used for evaluation, and GEE was used for analysis.

The experimental results showed that in Embodiment 1, the subjects did not experience any symptoms of gastrointestinal adverse reactions, and in comparative group 1, the subjects experienced diarrhea and abdominal distension. At the same time, compared with the sample in Embodiment 1, the β-casein-7 levels of serum interleukin-4, immunoglobulins G, E, and G1, as well as low glutathione in the subjects who consumed the sample of the comparative group 1 were significantly increased.

What is claimed is:

1. A hypoallergenic milk with immune enhancing function, comprising every 1000 kg of hypoallergenic milk contains 809-908 kg of raw milk, 24.7-42.2 kg of collagen peptide, 0.5-1.0 kg of yeast β-glucan, 5.7-29.5 kg of acerola cherry powder, 5-11 kg of maltitol, 0.1-1.3 kg of lactase, 1-4 kg of stabilizer, β-2 kg of food essence, and 55-100 kg of water, wherein the raw milk is A2β-casein organic raw milk.

2. The hypoallergenic milk with immune enhancing function according to claim 1, wherein each 250 mL of the hypoallergenic milk contains not more than 1.25 g of lactose, not less than 12.6 g of protein, not less than 96 mg of vitamin C, and not less than 87.5 mg of β-glucan.

3. The hypoallergenic milk with immune enhancing function according to claim 1, wherein a protein content of the A2β-casein organic raw milk is not be less than 3.12 g/100 g, and a β-casein content of the A2β-casein organic raw milk is not less than 0.9 g/100 g.

4. The hypoallergenic milk with immune enhancing function according to claim 3 wherein the collagen peptide is sourced from bovine bone with a protein content not less than 90 g/100 g.

5. The hypoallergenic milk with immune enhancing function according to claim 4, wherein a vitamin C content of the acerola cherry powder is not less than 16 g/100 g.

6. The hypoallergenic milk with immune enhancing function according to claim 5, wherein the lactase is sterile with an enzyme activity not less than 7500 U/g.

7. The hypoallergenic milk with immune enhancing function according to claim 6, wherein the stabilizer is composed of microcrystalline cellulose, gellan gum, carrageenan, and sodium carboxymethyl cellulose, and the mass ratio of the microcrystalline cellulose, the gellan gum, the carrageenan, and the sodium carboxymethyl cellulose is 0.3-1.7:0.4-0.9:0.2-0.8:0.1-0.6.

8. A preparation method of hypoallergenic milk with immune enhancing function according to claim 1, comprising the following steps:
step 1, conducting degassing, centrifugal clarification, homogenization, and pasteurization to the raw milk in sequence to obtain pasteurized milk, then refrigerating and storing at a temperature of 2-6° C.;
step 2, taking some of the pasteurized milk obtained from step 1, rising the temperature to 55-70° C., adding the collagen peptide, the yeast β-glucan, the maltitol, and the stabilizer in a stirred state, and mixing and dissolving for 10-15 minutes;
step 3, accurately weighing the acerola cherry powder to adding into pre-weighed normal temperature water to obtain acerola cherry powder solution, then adding the acerola cherry powder solution to the material solution obtained in step 2 after the acerola cherry powder is stirred and fully dissolved, then continuously stirring for 5-10 minutes to mix and dissolve all raw and auxiliary materials evenly;
step 4, filtering and cooling the material solution obtained from step 3 below 20° C., mixing the cooled material solution with a remaining pasteurized milk for 15-20 minutes, diluting to volume and then stirring for 15-20 minutes, and then conducting homogenization and sterilization, wherein a pressure of the homogenization is 20±5 MPa, a temperature of the homogenization is 65±5° C.; a temperature of ultra-high temperature sterilization is 135-150° C., and time of the ultra-high temperature sterilization is 2-15 seconds;
step 5, lowering the temperature of the material solution obtained in step 4 to below 25° C., then adding sterile lactase, and then conducting aseptic filling.

9. The preparation method of hypoallergenic milk with immune enhancing function according to claim 8, wherein a temperature of the pasteurization in step 1 is 75-85° C. and the time is 10-15 seconds.

10. The preparation method of hypoallergenic milk with immune enhancing function according to claim 9, wherein adding the food essence before diluting to volume in step 4.
